(19) United States
(12) Patent Application Publication
(10) Pub. No.: US 2026/0306034 A1
(43) Pub. Date: Oct. 8, 2026

(54) FLOOR ASSEMBLY FOR PATIENT POSITIONING SYSTEM

(71) Applicant: LEO CANCER CARE, INC., Middleton, WI (US)

(72) Inventors: Mark Strangeman, West Sussex (GB); Ralf Spriesterbach, Athens (GR); Brent Harper, New Glarus, WI (US)

(21) Appl. No.: 19/479,917

(22) PCT Filed: May 7, 2024
(86) PCT No.: PCT/US2024/028106

§ 371 Date: Oct. 29, 2025

Related U.S. Application Data
(60) Provisional application No. 63/464,684, filed on May 8, 2023.

(51) Int. Cl. A61B 6/04; A61B 6/03

(52) U.S. Cl. CPC A61B 6/0487; A61B 6/0478; A61B 6/032

(57) ABSTRACT

A patient positioning system including an opening formed in a floor, a carriage movable with respect to the opening, and a stack of plates coupled to the carriage. The stack of plates includes a first plate, a second plate, a spacer positioned between the first plate and the second plate, and a key coupled between the first plate and the second plate. The first plate is slidable with respect to the second plate.

### STATEMENT OF RELATED APPLICATIONS

This application claims the benefit of U.S. Provisional Patent Application No. 63/464,684, filed May 8, 2024, the entire contents of which are incorporated herein by reference for all purposes.

### FIELD

Provided herein is technology relating to radiology and radiotherapy and particularly, but not exclusively, to apparatuses, methods, and systems for medical imaging.

### BACKGROUND

Medical imaging is used to diagnose, stage, plan treatment, guide treatment, and evaluate response to treatment in patients for numerous types of diseases, injuries, and other maladies. In particular, computerized tomography (CT) is a form of medical imaging that produces a three-dimensional model of an object (e.g., a patient or portion thereof) using multiple two-dimensional X-ray measurements taken from different angles. CT imaging produces tomographic (cross-sectional) images of targeted areas of a patient or portion thereof, thus allowing a user to image the interior of the patient without cutting the patient. In conventional CT, a patient is placed horizontally on a couch or gurney and the patient and couch are moved into the CT scanning apparatus. Alternatively, gurneys may be fixed and the CT scanner moves horizontally. New technologies are needed to allow imaging of patients safely in multiple positions, e.g., vertical and/or essentially vertical positions (e.g., standing, sitting, kneeling, etc.) in addition to horizontal positions and/or essentially horizontal positions (e.g., lying (e.g., prone or supine)) and other patient positions such as tilted forwards or backwards and other orthopedic positions.

### SUMMARY

The technology described herein relates to medical treatment and medical imaging, e.g., computerized tomography (CT), magnetic resonance imaging (MRI), positron emission tomography (PET), single photon emission computerized tomography (SPECT), photon counting computed tomography, portal imaging (e.g., prior to a treatment), radiograph 1 localizers, topograms or scanned projection radiography (“scout view”) (e.g., prior to an imaging scan and/or prior to a treatment).

The technology provided herein relates to a patient positioning system for medical imaging and/or radiation therapy. In some embodiments, the technology described herein supplements and/or modifies a patient positioning apparatus and/or a patient support, e.g., as described in U.S. Pat. App. Pub. No. 20200268327, which is incorporated herein by reference. In some embodiments, the patient positioning system stabilizes and supports a patent in an upright (e.g., standing, sitting, kneeling, perched) position. Imaging and/or treating patients in an upright position provides the benefits of increasing patient comfort. Further, diagnosis and/or treatment of patients in an upright position provides advantages over conventional diagnosis and/or treatment of patients in a horizontal position for many indications (e.g., lung cancer, breast cancer). While imaging and/or treating an upright patient provides diagnostic and therapeutic advantages, medical imaging and treatment needs improved patient positioning systems for stabilizing and supporting patients in a suitable upright position for delivering therapeutic radiation doses to target areas and for planning treatment using medical imaging.

The disclosure provides, in one aspect, a patient positioning system including an opening formed in a floor, a carriage movable with respect to the opening, and a stack of plates coupled to the carriage. The stack of plates includes a first plate, a second plate, a spacer positioned between the first plate and the second plate, and a key coupled between the first plate and the second plate. The first plate is slidable with respect to the second plate.

In some embodiments, the key is positioned within a slot formed in the second plate, and the key is fastened to the first plate.

In some embodiments, the spacer is positioned within a groove formed in the second plate.

In some embodiments, the slot is on a first side of the second plate and the groove is on a second side of the second plate.

In some embodiments, the carriage includes a drive plate, and the drive plate is coupled to the first plate.

In some embodiments, the opening is entirely covered.

In some embodiments, the stack of plates further includes a third plate, wherein the second plate is positioned between the first plate and the third plate.

In some embodiments, the key is a first key, and the stack of plates further including a second key coupled between the second plate and the third plate.

In some embodiments, the first key is offset from the second key.

In some embodiments, the spacer at least partially overlaps the key.

In some embodiments, the spacer is positioned within a groove formed in the second plate.

In some embodiments, the patient positioning system further includes a corner plate coupled to the stack of plates and configured to move between a retracted position and an extended position.

In some embodiments, the patient positioning system further includes a cover at least partially defining the opening, and wherein the corner plate is positioned between the stack of plates and the cover.

In some embodiments, the stack of plates is a first stack of plates, and the patient positioning system further includes a second stack of plates.

In some embodiments, the carriage is positioned between the first stack of plates and the second stack of plates.

In some embodiments, the first stack of plates and the second stack of plates adjusts in response to movement of the carriage within the opening.

In some embodiments, a first corner plate is coupled to the first stack of plates and a second corner plate is coupled to the second stack of plates.

In some embodiments, a cover is at least partially defining the opening, wherein the cover has a circular outer profile.

In some embodiments, the patient positioning system further includes a patient support coupled to the carriage.

In some embodiments, the patient positioning system further includes an actuator configured to move the carriage, wherein the actuator is positioned below the floor.

The disclosure provides, in one aspect, a patient positioning system comprising a floor assembly including: a first floor piece with a slot; a second floor piece with an aperture positioned offset from a center of the of the second floor piece; and a third floor piece positioned within the aperture. The patient positioning system further includes a patient support coupled to the third floor piece.

In some embodiments, the third floor piece defines an axis and the third floor piece is rotatable about the axis with respect to the first floor piece.

In some embodiments, the third floor piece is positioned within the slot.

In some embodiments, the third floor piece is movable within the slot relative to the first floor piece.

In some embodiments, the second floor piece is rotatable with respect to the first floor piece and the third floor piece.

In some embodiments, the third floor piece includes a lip and the second floor piece is at least partially positioning on the lip.

In some embodiments, the patient positioning system further includes a support assembly coupled to the floor assembly, wherein the support assembly includes an outer frame, an inner frame coupled to the second floor piece, and a plurality of brackets positioned between the inner frame and the outer frame.

In some embodiments, the inner frame is an inner ring and the outer frame is an outer ring.

In some embodiments, each of the plurality of brackets is angled.

In some embodiments, each of the plurality of brackets is pivotably coupled to the inner frame at a first end and pivotably coupled to the outer frame at a second end opposite the first end.

In some embodiments, the patient positioning system further includes an actuator coupled to the second floor piece and configured to move the second floor piece.

In some embodiments, the first floor piece is at least partially positioned on the second floor piece.

In some embodiments, the patient positioning system further includes a first linear translation stage coupled to the third floor piece and configured to move the third floor piece along a first translation axis and a second linear translation stage coupled to the third floor piece and configured to move the third floor piece along a second translation axis.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings. The patent or application file contains at least one drawing executed in color. Copies of this patent or patent application publication with color drawings will be provided by the Office upon request and payment of the necessary fee.

FIG. 1 a perspective view of a patient positioning system.

FIG. 2 is a perspective view of an opening formed in a floor.

FIG. 3 is a perspective view of the patient positioning system of FIG. 1, with portions removed for clarity.

FIG. 4 is a perspective view of FIG. 3, with portions removed for clarity.

FIG. 5 is a bottom perspective view the patient positioning system of FIG. 3.

FIG. 6 is a perspective cross sectional view of the patient positioning system of FIG. 3.

FIG. 7 is a partial perspective cross sectional view of the patient positioning system of FIG. 3, illustrating an adjustable stack of plates.

FIG. 8 is a partial exploded view the adjustable stack of plates of the patient positioning system of FIG. 3.

FIG. 9 is another partial exploded view of the adjustable stack of plates of the patient positioning system of FIG. 3.

FIG. 10 is a partial top perspective cross-sectional view of the patient positioning system of FIG. 3, illustrating a corner plate.

FIG. 11 is a partial bottom perspective cross sectional view of the patient positioning system of FIG. 3, illustrating the corner plate.

FIG. 12A is a perspective view of a patient positioning system shown in a centered position.

FIG. 12B is a perspective view of the patient positioning system of FIG. 12A, shown in an intermediate position.

FIG. 12C is a perspective view of the patient positioning system of FIG. 12A, shown in a maximum translation position.

FIG. 13 is a cross sectional view of the patient positioning system of FIG. 12A.

FIG. 14 is a perspective view of the cross-section of FIG. 13.

FIG. 15 is a perspective view of a floor assembly for the patient positioning system of FIG. 12A.

FIG. 16 is a top view of a main floor piece, a secondary floor piece, and a central floor piece for the floor assembly of FIG. 15.

FIG. 17 is a side view of the main floor piece, the secondary floor piece, and the central floor piece of FIG. 16.

FIG. 18A is a bottom view of a support assembly for the secondary floor piece, shown in a centered position.

FIG. 18B is a bottom view of the support assembly and the secondary floor piece of FIG. 18A, shown in an intermediate position.

FIG. 18C is a bottom view of the support assembly and the secondary floor piece of FIG. 18, shown in a maximum translation position.

FIG. 19A is a bottom view of the support assembly and the floor assembly, shown in a centered position.

FIG. 19B is a bottom view of the support assembly and the floor assembly, shown in an intermediate position.

FIG. 19C is a bottom view of the support assembly and the floor assembly, shown in a maximum translation position.

FIG. 20 is a cross-sectional partial view of the support assembly and the floor assembly.

FIG. 21A is a perspective view of the support assembly in a centered position.

FIG. 21B is a top view of the support assembly of FIG. 21A.

FIG. 22A is a perspective view of the support assembly in a maximum translation position.

FIG. 22B is a top view of the support assembly of FIG. 22A.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to medical imaging and radiation therapy and particularly, but not exclusively, to devices, methods, and systems for positioning and supporting a patient with respect to a radiation source to image the patient and/or to treat the patient by exposing the patient to a radiation beam produced by the radiation source.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling, performing, and/or directing methods.

As used herein, the term “computed tomography” is abbreviated “CT” and refers both to tomographic and non-tomographic radiography. For instance, the term “CT” refers to numerous forms of CT, including but not limited to X-ray CT, positron emission tomography (PET), single photon emission computed tomography (SPECT), and photon counting computed tomography. Generally, computed tomography (CT) comprises use of an X-ray source and a detector that rotates around a patient and subsequent reconstruction of images into different planes. Currents for X-rays used in CT describe the current flow from a cathode to an anode and are typically measured in milliamperes (mA).

As used herein, the term “coupled” refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components. As used herein, “directly coupled” means that two elements are directly in contact with each other. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first clement being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the term “operatively coupled” means that a number of elements or assemblies, each of which is movable between a first position and a second position, or a first configuration and a second configuration, are coupled so that as the first element moves from one position/configuration to the other, the second element moves between positions/configurations as well. It is noted that a first element may be “operatively coupled” to another without the opposite being true.

As used herein, the term “slidably coupled” refers to two or more components that are coupled in a manner such that at least one of the components is slidable with respect to the other.

As used herein, the term “correspond” indicates that two structural components are sized and shaped to be similar to each other and may be coupled with a minimum amount of friction. Thus, an opening which “corresponds” to a member is sized slightly larger than the member so that the member may pass through the opening with a minimum amount of friction. This definition is modified if the two components are to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines, two, or more, “corresponding” surfaces, shapes, or lines have generally the same size, shape, and contours.

As used herein, in the phrase “[x] moves between its first position and second position,” or, “[y] is structured to move [x] between its first position and second position,” “[x]” is the name of an element or assembly. Further, when [x] is an element or assembly that moves between a number of positions, the pronoun “its” means “[x],” e.g., the named element or assembly that precedes the pronoun “its.”

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. For instance, the term “subject” or “patient” refers to organisms including, but not limited to, humans and veterinary animals (dogs, cats, horses, pigs, cattle, sheep, goats, and the like). In the context of the technology, the term “subject” or “patient” generally refers to an individual who will be subject to a CT scan to diagnose a disease or injury; and/or to prepare for a treatment.

As used herein, a “diagnostic” test includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject having a cancer or the likelihood that such a subject will respond favorably to a compound (e.g., a pharmaceutical, e.g., a drug) or other treatment.

As used herein, the term “condition” refers generally to a disease, malady, injury, event, or change in health status.

As used herein, the term “treating” or “treatment” with respect to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, “treatment” comprises exposing a patient or a portion thereof (e.g., a tissue, organ, body part, or other localize region of a patient body) to radiation (e.g., electromagnetic radiation, ionizing radiation).

The term “network” as used herein generally refers to any suitable electronic network including, but not limited to, a wide area network (“WAN”) (e.g., a TCP/IP based network), a local area network (“LAN”), a neighborhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc.

The term “computer” as used herein generally includes a plurality of electrical and electronic components that provide power, operational control, and protection to the components and modules within the system. For example, a computer can include, among other things, a processing unit (e.g., a microprocessor, a microcontroller, or other suitable programmable device), a memory, input units, and output units. The processing unit can include, among other things, a control unit, an arithmetic logic unit (“ALC”), and a plurality of registers, and can be implemented using a known computer architecture (e.g., a modified Harvard architecture, a von Neumann architecture, etc.). A “microprocessor” or “processor” refers to one or more microprocessors that can be configured to communicate in a stand-alone and/or a distributed environment, and can be configured to communicate via wired or wireless communications with other processors, where such one or more processors can be configured to operate on one or more processor controlled devices that can be similar or different devices.

The term “memory” as used herein generally refers to any memory storage of the computer and is a non transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processing unit can be connected to the memory and execute software instructions that are capable of being stored in a RAM of the memory (e.g., during execution), a ROM of the memory (e.g., on a generally permanent bases), or another non transitory computer readable medium such as another memory or a disc. “Memory” can include one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the computer can be configured to retrieve from the memory and execute, among other things, instructions related to the processes and methods described herein.

### Description

Although the disclosure herein refers to certain illustrated embodiments, it is to be understood that these embodiments are presented by way of example and not by way of limitation.

The technology provided herein relates to medical treatment and/or medical imaging apparatus. While described in some embodiments for computed tomography (CT), the technology is not limited to use with CT and finds use for other medical imaging technologies such as, e.g., radiography, fluoroscopy, MRI, SPECT, PET, photon counting computed tomography, and portal imaging (e.g., prior to a treatment) or scanned projection radiography. Computed tomography (CT), and in particular computer X-ray tomography, is an imaging technique that generates cross-sectional images of a patient by mathematically combining multiple X-ray images (projections) taken along the plane of the cross-section at 12 a range of angles. In conventional CT, generating a tomographic image involves providing a projection set of multiple projections over at least 180 degrees and preferably 360 degrees of angular range about the patient. The patient is typically moved through a gantry holding an X-ray source and X-ray detector that turn in coordinated opposition about the patient to acquire each X-ray projection set, either continuously during the orbital motion (helical scanning) or stepwise in between orbits (step scanning) to obtain X-ray projection sets for adjacent cross-sectional images that together describe a volume of tissue. Movement of the patient is conventional CT is provided by supporting a horizontal patient on a horizontally extending radio translucent table that is moved through the gantry.

CT imaging of some patients may preferably be performed with the patient in a vertical position (e.g., a sitting, kneeling, standing, and/or reclining position (e.g., seated, seated and leaning backward, seated and leaning forward, standing, standing and leaning backward, standing and leaning forward, kneeling, kneeling and leaning forward, or kneeling and leaning backward)). For example, a lung cancer patient undergoing thoracic radiotherapy may prefer to be in a standing position so as not to promote the coughing that often accompanies this treatment. Some medical conditions such as vertebral fractures may be more evident in a weight-bearing standing position. Accordingly, CT scanners that record CT scans of patients in a vertical position would benefit medical diagnosis and treatment. Further, a CT scanner that is capable of scanning on multiple axes, e.g., to scan patients in a vertical position, patients positioned in a conventional horizontal position, and in other positions, would expand the use scenarios of the CT scanner to address more diseases, injuries, and maladies, and to improve the cost effectiveness of the CT scanner.

In some embodiments, the medical treatment apparatus includes a therapy beam and does not include an imaging beam.

### Apparatus

In some embodiments, the technology relates to a multi-axis medical imaging apparatus. In some embodiments, the medical imaging apparatus is a computerized tomography (CT) apparatus, a magnetic resonance imaging (MRI) apparatus, a positron emission tomography (PET) apparatus, a single photon emission computerized tomography (SPECT) apparatus, a photon counting computed tomography apparatus, or a portal imaging or scan projection radiography apparatus. While the technology is described for exemplary embodiments wherein the medical imaging apparatus is a computerized tomography (CT) apparatus, the technology is not limited to a CT scanning apparatus and embodiments are to be understood to include other types of medical imaging apparatuses, methods, and systems. In other embodiments, the technology relates to a multi-axis medical treatment apparatus.

In some embodiments, the technology provides a multi-axis CT scanner as described in U.S. Patent Application Publication No. 2022/0183641, filed Nov. 24, 2021, which is incorporated herein by reference. In some embodiments, the patient is positioned vertically. In some embodiments, a patient that is positioned vertically is positioned with a slight recline (e.g., within 20 degrees of vertical (e.g., within 5, 6, 7, 8, 9, 10, 11, 12, 13, 14, 15, 16, 17, 18, 19, or 20 degrees)) so that the patient may lean against a surface for support to provide increased immobilization of the patient and to limit motion of the patient. In some embodiments, the patient is positioned using a patient positioning system and the user operates a control unit. In some embodiments, the patient positioning system is as described in Int'l Pat. App. Pub. No. WO 2019/056055 and U.S. Pat. App. Pub. No. 2020/0268327, each of which is incorporated herein by reference.

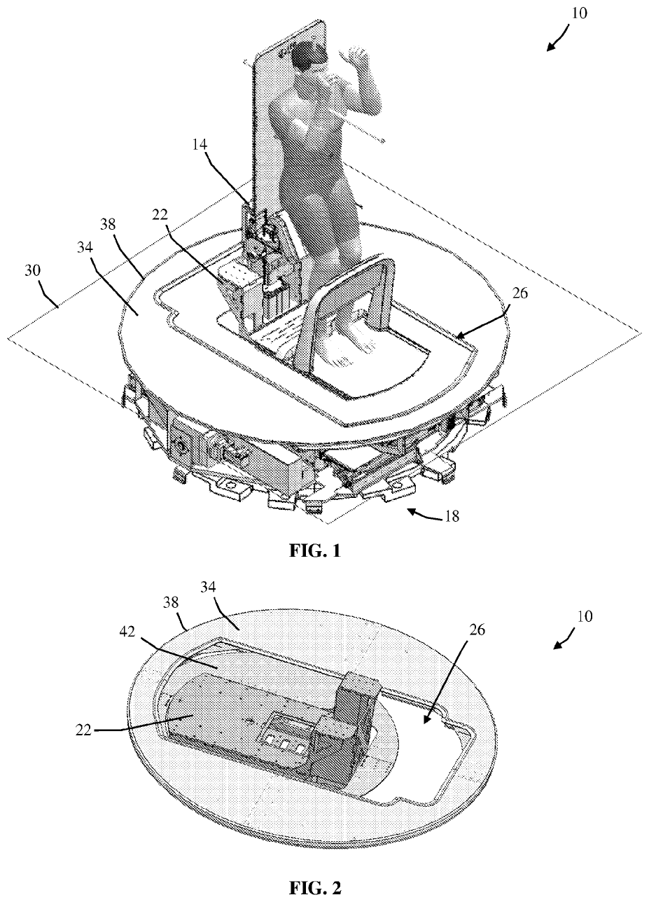

With reference to FIG. 1, a patient positioning system 10 comprises a configurable patient support 14 (e.g., patient support, patient support assembly, etc.). In some embodiments, the patient support 14 comprises a generally elongate structure. The patient support 14 is configured to receive and secure a patient in a generally upright position. The patient support 14 is rotatably mounted to a translatable member such that the patient support is rotatable about a vertical axis (e.g., an essentially vertical and/or substantially vertical axis) relative to the translatable member. A lower end of the patient support is mounted to a rotating disc. In some embodiments, an upper end of the patient support is mounted to another rotating disc. By this arrangement, the patient support is rotatably mounted to the translatable member such that the patient support is rotatable about a vertical axis. Also, as the translatable member is able to articulate vertically, the patient support mounted to the translatable member may similarly articulate vertically.

The patient support 14 is adapted to receive a patient requiring exposure to a treatment beam and/or to an imaging beam. The patient support is offset from the vertical axis of rotation such that the torso of a patient secured to the patient support is aligned with the vertical axis of rotation (e.g., a vertical axis of the patient is aligned with the vertical axis of rotation). Accordingly, in some embodiments, the position of a patient supported by the patient support may be adjusted by the rotational coupling of the patient support to the translatable member and/or by the vertical translation of the translatable member. By this arrangement, the position of a patient may be adjusted with respect to a treatment beam or an imaging beam so that the treatment beam or imaging beam may target the area of the patient requiring treatment or imaging.

For example, in some embodiments, a standing patient is supported by the patient support when the translatable member is in the first position. Further, in some embodiments, a standing patient is supported by the patient support when the translatable member is in a vertically upwards position termed the second position. When the translatable member is in the second position, the fixed treatment beam may be directed towards the prostate of the patient, for example. In some embodiments, the translatable member is moved to an appropriate vertical position between the first position and the second position to align the fixed treatment beam with a section of the patient between the patient feet and navel. Accordingly, the technology provides for the fixed treatment beam to target any part of a patient, e.g., from the top of the head to the prostate, while minimizing the vertical travel of the translatable member. Accordingly, the patient positioning system provides a patient support appropriate for standing and sitting patients of all heights, e.g., a range of heights from a patient in the 95th height percentile of an American male to a patient in the 5th height percentile of a Japanese female. In some embodiments, the translatable member vertically translates approximately 3000 to 4000 mm (e.g., 3000, 3050, 3100, 3150, 3200, 3250, 3300, 3350, 3400, 3450, 3500, 3550, 3600, 3650, 3700, 3750, 3800, 3850, 3900, 3950, or 4000 mm) to target any portion from head to foot of a 95th height percentile American male patient. One advantage of minimizing the vertical travel of the translatable member is that the vertical space required by the patient positioning system is minimized, thus allowing the patient positioning system to be located in a space smaller than conventional treatment bunkers and reducing the costs of installation and assembly.

When the part of a patient requiring treatment may be targeted in either of the standing or seated positions, the position in which the patient is supported by the patient support is selectable (e.g., based on patient comfort, patient condition, and/or treatment protocol).

In some embodiments, the patient support 14 comprises a back rest, a seat member, a shin rest, an arm rest, a head rest, and/or foot braces. Embodiments provide that the back rest, seat member, shin rest, arm rest, a head rest, and/or foot braces is/are configurable among a number of positions to accommodate patient ingress and/or egress from the patient support system (e.g., from the patient support assembly) and/or to support a patient in a number of positions for imaging or treatment. In some embodiments, the patient support 14 is a chair or a table.

As described herein, embodiments provide that the patient support 14 is mounted to a rotatable structure, e.g., a rotating disc of a translatable member, to provide rotation of the patient support assembly about a vertical axis. For example, in some embodiments, a platform is mounted to the rotating disc. In some embodiments, the platform is integrally formed with the rotating disc and forms part of the rotating disc.

In some embodiments, the patient support 14 is configured to move or translate along a vertical direction and multiple horizontal directions. For example, in some embodiments, the patient support assembly is mounted to and above an actuator 18. Accordingly, the vertical and/or horizontal location of the patient support assembly and of the patient may be adjusted without affecting the configuration of the patient support assembly or the position of the patient relative to the patient support assembly. In some embodiments, the patient support assembly and the translatable member share the same vertical axis.

In some embodiments, the patient support 14 is translatable in a horizontal plane, e.g., in addition to being rotatable about a vertical axis. In some embodiments, the patient support 14 is translatable in a horizontal plane orthogonal to the vertical axis of rotation. In some embodiments, the patient support 14 is coupled to a carriage 22 (e.g., a translatable member) that is coupled to pairs of parallel rails in orthogonal relation, the carriage 22 being slidably connected to a first pair of rails for translation in a first orthogonal direction and the first set of rails being slidably connected to a second pair of rails for translation in a second orthogonal direction.

In some embodiments, the patient support 14 and/or the carriage 22 is configured to move with six degrees of freedom and further includes active compensation in at least one degree of freedom while another degree of freedom is adjusted. In some embodiments, the active compensation is utilized to maintain a desired patient region (e.g., a treatment zone, an imaging zone) aligned with a treatment or imaging beam, as described in U.S. patent application Ser. No. 17/894,335, filed Aug. 24, 2022, incorporated herein by reference in its entirety. In other words, the compensation ensures the patient treatment zone is aligned with the isocenter.

With reference to FIG. 2, portions of a patient positioning system 10 are illustrated. An opening 26 is formed in a floor 30 (e.g., a platform, a top surface, etc.) and the carriage 22 is movable with respect to the opening 26. In other words, there is space for the carriage 22 to move within the opening 26. This space, while necessary to provide relative movement between the carriage 22 and the floor 30, creates an undesirable opening in the floor. In addition, the shape of the space that is left unoccupied by the carriage 22 varies as the carriage 22 moves relative to the floor 30. In other words, the opening 26 created is a variable shape opening that is desirable to cover without impeding movement of the carriage 22 or the patient support 14. In the illustrated embodiment, the floor 30 includes a cover 34 at least partially defines the opening 26. The cover 34 has a circular outer profile 38. In some embodiments, a drive plate 42 is mechanically coupled to and moveable with the carriage 22. As described herein, the actuator 18 is positioned below the floor 30 and is configured to move the carriage 22.

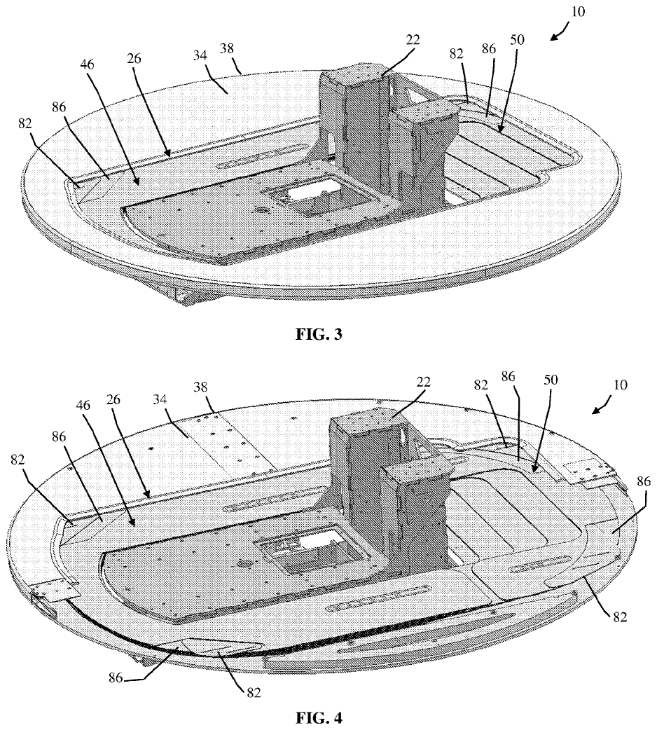

With reference to FIGS. 3 and 4, the patient positioning system 10 further includes a first stack of plates 46 (e.g., axial stack, plate stack, etc.) coupled to the carriage 22. In the illustrated embodiment, the patient positioning system 10 further includes a second stack of plates 50 coupled to the carriage 22. The first stack of plates 46 is a forward stack, and the second stack of plates 50 is a rearward stack. In other embodiments, a single stack of plates is provided. In other embodiments, more than two stacks of plates are provided (e.g., three stacks, four stacks, etc.). In the illustrated embodiment, the first stack of plates 46 is coupled to and at least partially surrounds a front portion of the carriage 22 and the second stack of plates 50 is coupled to and at least partially surrounds a rear portion of the carriage 22. In other words, the carriage 22 is positioned between the first stack of plates 46 and the second stack of plates 50.

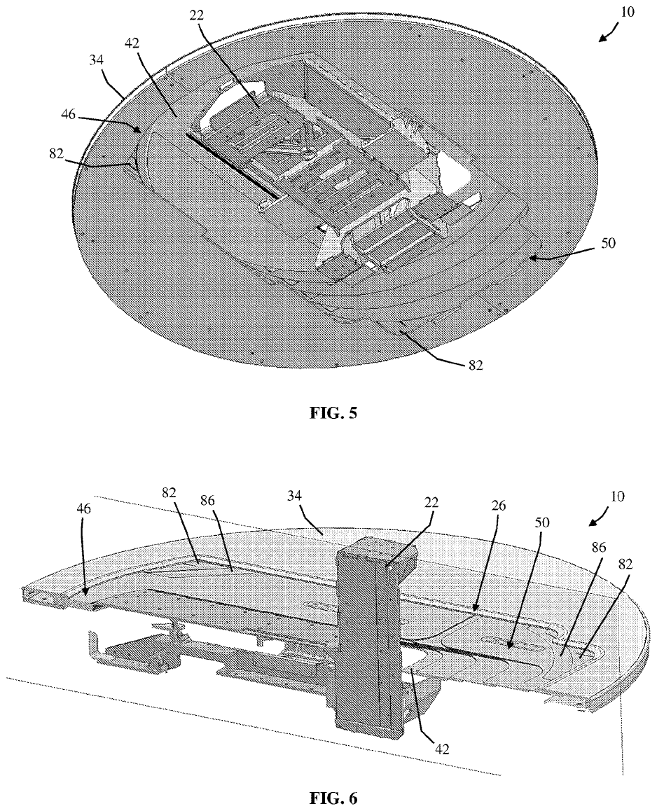

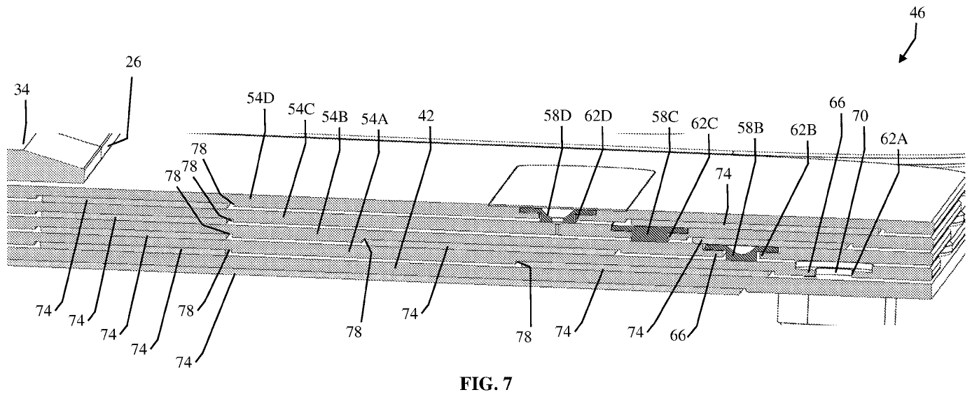

With reference to FIGS. 5 and 6, the drive plate 42 couples the carriage 22 to the stacks of plates 46, 50 (e.g., mechanically interconnects the carriage 22 to the stacks of plates 46, 50). In other words, the drive plate 42 is positioned between the carriage 22 and the first stack of plates 46 and is positioned between the carriage 22 and the second stack of plates 50. As discussed in further detail herein, the first stack of plates 46 and the second stack of plates 50 adjust in response to movement of the carriage 22 within the opening 26 such that the opening 26 remains advantageously covered regardless of the carriage 22 position.

Details of the first stack of plates 46 are described in further detail herein and such details are similarly applicable to the second stack of plates 50 in the illustrated embodiment.

With reference to FIG. 7, the first stack of plates 46 includes at least two plates. In the illustrated embodiment, the first stack of plates 46 includes a first plate 54A, a second plate 54B, a third plate 54C, and a fourth plate 54D. The first plate 54A is slidably coupled to the drive plate 42, the second plate 54B is slidably coupled to the first plate 54A, the third plate 54C is slidably coupled to the second plate 54B and the fourth plate 54D is slidably coupled to the third plate 54C. In other words, each of the plates 54A-54D is movable with respect to another one of the plates in the stack 46. In the illustrated embodiment, the second plate 54B is positioned between the first plate 54A and the third plate 54C, and the third plate 54C is positioned between the second plate 54B and the fourth plate 54D. In the illustrated embodiment, the stack of plates 46 is oriented such that the plates 54A-54D are stacked along a vertical direction.

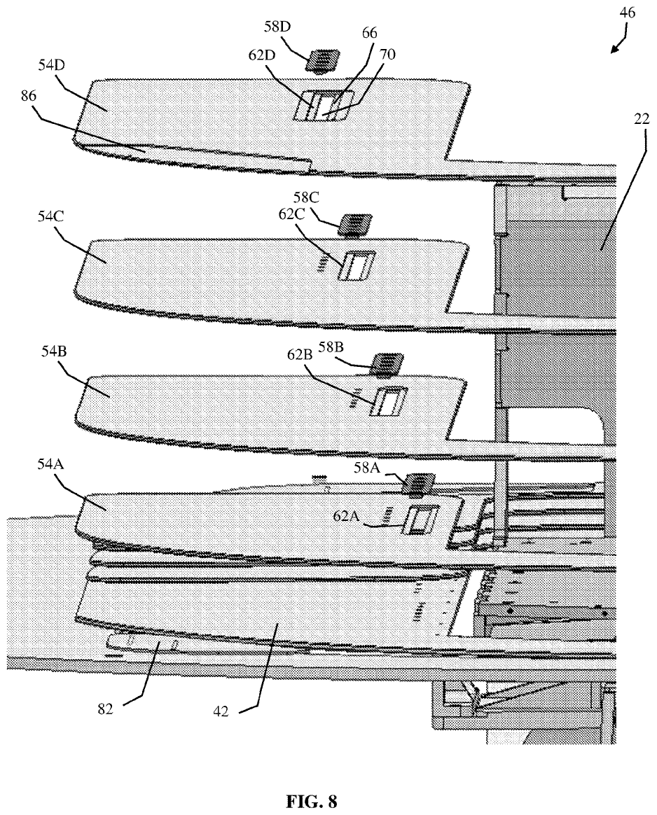

With reference to FIG. 8, the stack of plates 46 includes keys 54A, 54B, 54C, 54D that slidably couple adjacent plates. For example, a key 58B is coupled between the first plate 54A and the second plate 54B such that the first plate 54A is slidable with respect to the second plate 54B. A key 58A is coupled between the first plate 54A and the drive plate 42 such that the first plate 54A is slidable with respect to the drive plate 42. In the illustrated embodiment, the keys 58A-58D are positioned with a corresponding slot 62A-62D formed in one of the plates. For example, the key 58B is positioned within a slot 62B formed in the second plate 54B and the key 588 is fastened to the first plate 54A. In the illustrated embodiment, each of the slots 62A-62D includes a lip portion 66 and an open portion 70. In the illustrated embodiment, the lip portion 66 surrounds the open portion. A portion of the key 58B extends through the open portion 70, and a portion of the key 58B slides along the lip portion 66. Similarly, the key 58C coupled between the second plate 54B and the third plate 54C such that the second plate 54B is slidable with respect to the third plate 54C. The key 58C is positioned within the slot 62C formed in the third plate 54C and the key 58C is fastened to the second plate 54B. In the illustrated embodiment, the keys 58A-58D are offset from each other in a left right direction as viewed from the frame of reference of FIG. 8. For example, the key 58B and the key 58C are not vertically aligned.

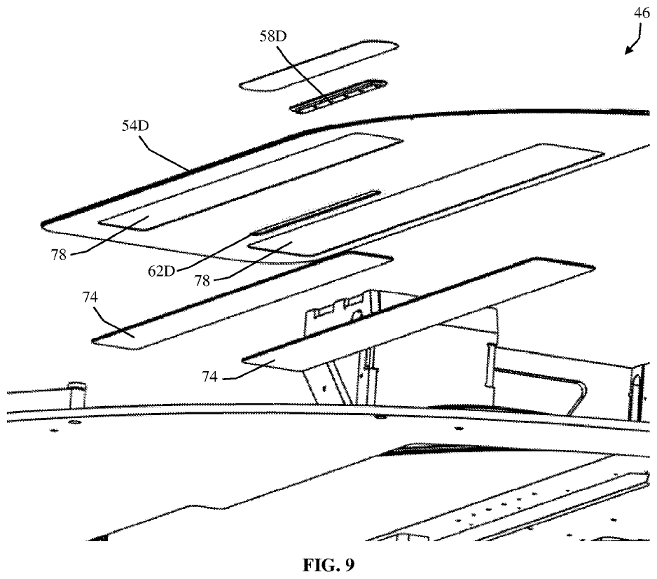

With reference to FIG. 9, at least one spacer 74 is positioned between adjacent plates (e.g., 54C and 54D) of the stack 46. The spacers 74 are received within grooves 78 formed in one of the plates. For example, with reference to FIG. 9, the spacers 74 are position within grooves 78 formed in the fourth plate 54D and are positioned between the fourth plate 54D and the third plate 54C. In some embodiments, the spacers 74 are made of a low-friction material (e.g., nylon). In the illustrated embodiment, each of the plates 54A-54D includes at least one slot (e.g., 62A-62D) to receive a key on a first side (e.g., a top side of the plate) and at least one groove 78 to receive a spacer 74 on a second side (e.g., a bottom side of the plate). In other words, the slots 62A-62D and the grooves 78 are positioned on opposite sides of the plates 54A-54D.

With reference to FIG. 7, in some embodiments, a spacer 74 at least partially overlaps a key (e.g., key 58B and 58C). In the illustrated embodiment, two spacers 74 are positioned between adjacent plates (e.g., 54B and 54A) and support the adjacent plates during relative translation.

In operation, as the carriage 22 is moved by the actuator 18, the drive plate 42 correspondingly moves the first and second stacks of plates 46, 50. The stacks of plates 46, 50 reconfigure the relative positions of the plates within the stack as the carriage 22 moves. For example, when the carriage 22 is in a forward most center position in the opening 26, the first stack of plates 46 is vertically stacked with the plates 54A-54D stacked one on top of another (similar to the stacked arrangement shown in FIG. 7). At the same time, the second stack of plates 50 is extended such that the individual plates in the stack are at least partially horizontally spaced from one another. In other words, as the carriage 22 and the patient support 14 moves within the opening 26, the stack of plates 46, 50 adjust to ensure the portions of the opening 26 not occupied by the carriage 22 remain covered. The stack of plates 46, 50 provide the structural strength to support the loading conditions that occur during operation (e.g., patient weight, technician weight, point load weights from a bed, for example, etc.).

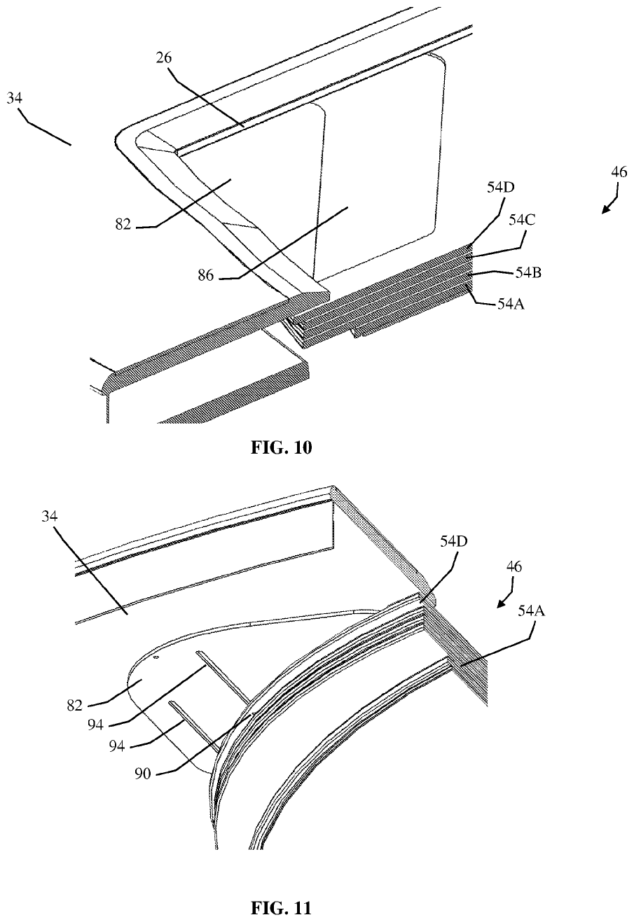

With reference to FIGS. 10 and 11, the patient positioning system 10 includes a corner plate 82 coupled to the stack of plates 46. The corner plate 82 is configured to move between a retracted position (in which the corner plate 82 does not extend into the opening 26) and an extended position (in which the corner plate 82 extends into the opening 26). In the illustrated embodiment, a pocket 86 is formed in the stack of plates 46 and the corner plate 82 is at least partially received within the pocket 86. In the illustrated embodiment, the corner plate 82 is positioned between the stack of plates 46 and the cover 34. In the illustrated embodiment, a pin 90 positioned on the top-most plate of the stack of plates 46 (e.g., the fourth plate 54D) is received within a groove 94 formed in the corner plate 82. With reference to FIG. 4, the illustrated patient positioning system 10 includes four corner plates 82 with one positioned generally in each corner of the opening 26. In particular, two corner plates 82 are coupled to the first stack of plates 46 and two corner plates 82 are coupled to the rear stack of plates 50.

In operation, when the carriage 22 is positioned in an end of travel position (such as a corner of the opening 26) the corner plates 82 in other corners move to the extended position to extend into the opening 26. In other words, the corner plates 82 are deployed from the retracted position to the extended position automatically when carriage 22 moves the stack of plates 46 into a position where the stack of plates 46 alone would not cover the opening 26 at the corresponding corner. In other words, the corner plates 82 provide coverage of the opening 26 when the carriage 22 is in an end of travel position and allows the overall size of the stack of plates 46, 50 to remain small and not need to be over sized for those end-of travel positions.

Advantageously, the opening 26 is entirely covered regardless of the position of the patient support 14 for improved safety, for example. In the illustrated embodiment, components that entirely cover the opening 26 include, but are not limited to, the first stack of plates 46 and the second stack of plates 50. In some embodiments, components that entirely cover the opening 26 includes, but are not limited to, a stack of plates and at least one corner plate 82. The design disclosed herein covers the opening 26 while maintaining the required strength and keeping an overall small footprint that is manageable and able to be installed into a floor.

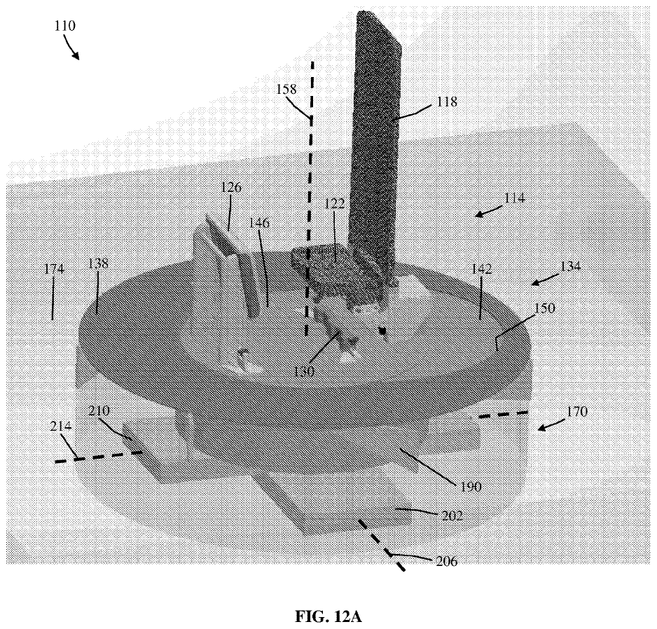

With reference to FIGS. 12A-12C, a patient positioning system 110 comprises a configurable patient support 114 (e.g., patient support, patient support assembly, etc.). In the illustrated embodiment, the patient support 114 comprises a back rest 118, a seat member 122, a shin rest 126, and a heel stop 130. The patient support 114 is configured to receive and secure a patient in a generally upright position. As detailed herein, the position of a patient may be adjusted with respect to a treatment beam or an imaging beam so that the treatment beam or imaging beam may target the area of the patient requiring treatment or imaging.

The patient positioning system 110 further includes a floor assembly 134. In the illustrated embodiment, the floor assembly 134 includes a first floor piece 138 (e.g., a main floor piece), a second floor piece 142 (e.g., a secondary floor piece), and a third floor piece 146 (e.g., a central floor piece). The patient support 114 is coupled to the floor assembly 134. In the illustrated embodiment, the patient support 114 is coupled to the third floor piece 146.

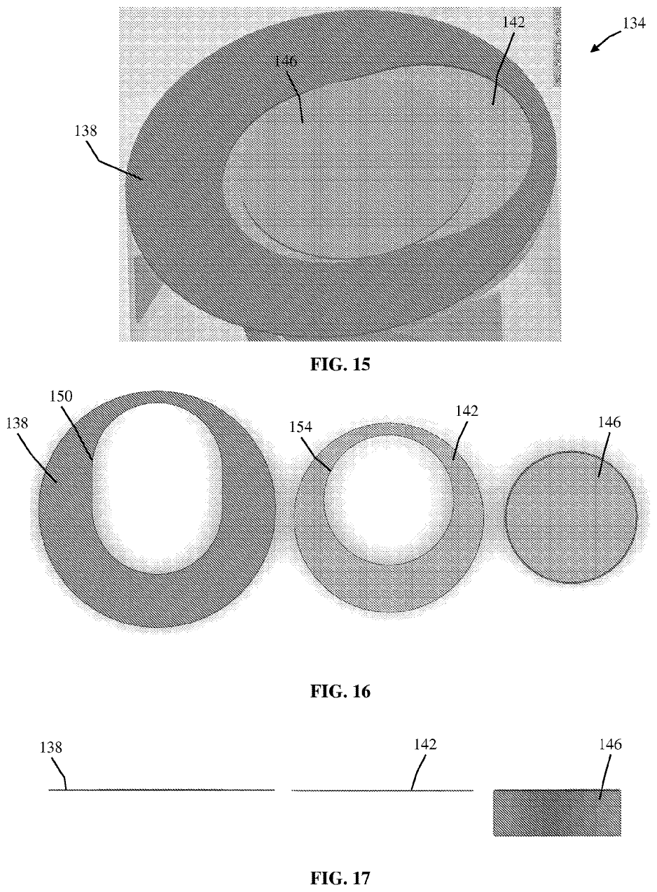

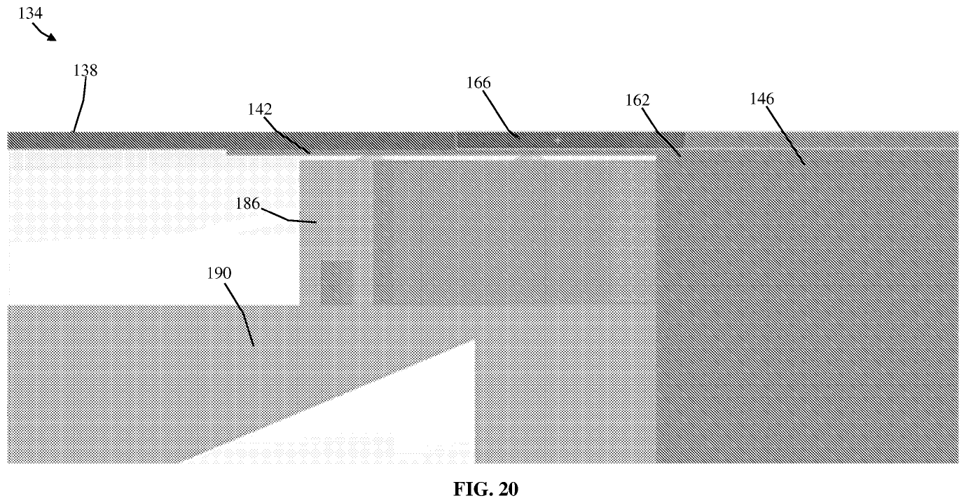

With reference to FIGS. 15, 16, and 17, the first floor piece 138 includes a slot 150. In some embodiments, the slot 150 comprises an approximately 1100 mm diameter opening cut into an approximately 350 mm long slot. In the illustrated embodiment, the first floor piece 138 is at least partially positioned on the second floor piece 142 (FIG. 20). In some embodiments, the first floor piece 138 is approximately 5 mm thick. In some embodiments, the first floor piece 138 has an outer diameter of approximately 2000 mm.

With continued reference to FIGS. 15, 16, and 17, the second floor piece 142 includes an aperture 154 positioned offset from a center of the second floor piece 142. In some embodiments, the aperture 154 has a diameter of approximately 1100 mm. In some embodiments, the aperture 154 is centered around the patient support 114. In the illustrated embodiment, the aperture 154 is offset from the outer perimeter of the second floor piece 142. In the illustrated embodiment, the second floor piece 142 is rotatable with respect to the first floor piece 138 and the third floor piece 146. As such, the second floor piece 142 fills in the gaps between the first floor piece 138 and the third floor piece 146. In some embodiments, the second floor piece 142 is approximately 5 mm thick.

With continued reference to FIGS. 15, 16, and 17, the third floor piece 146 is positioned within the aperture 154. In the illustrated embodiment, the third floor piece 146 is also positioned within the slot 150 of the first floor piece 138. In some embodiments, the third floor piece 146 has a diameter of approximately 1100 mm. In the illustrated embodiment, the third floor piece 146 is movable within the slot 150 relative to the first floor piece 138. The third floor piece 146 defines an axis 158. In the illustrated embodiment, the axis 158 is vertical. The third floor piece 158 is rotatable about the axis 158 with respect to the first floor piece 138.

With reference to FIG. 20, the third floor piece 146 includes a lip 162 and the second floor piece 142 is at least partially positioned on the lip 162. When assembled, the floor assembly 134 advantageously only includes a single recess 166 of approximately 5 mm. In other words, the floor assembly 134 provides movable complete coverage of the floor for dust and human support with only the single recess 166, which is a significant improvement from conventional solutions. Advantageously, the floor pieces 138, 142, 146 of the floor assembly 134 can be implemented with thin, flat, pieces that are easy to fabricate to tight tolerances and at low costs.

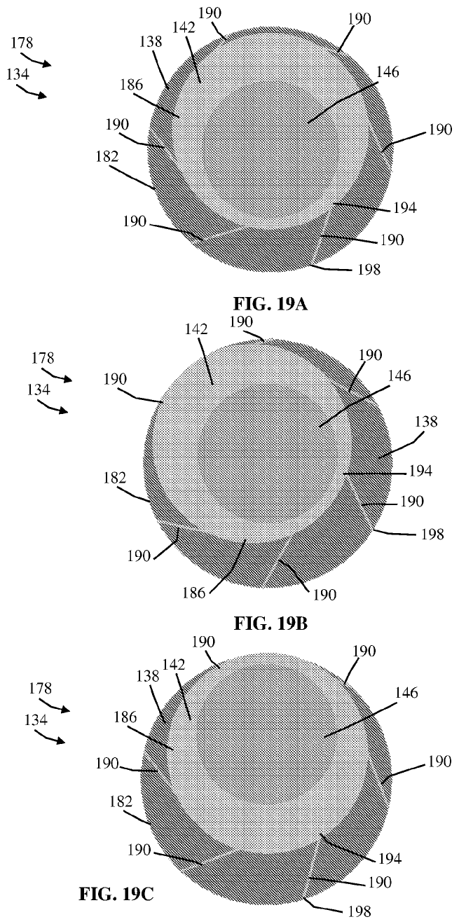

In the illustrated embodiment, the floor pieces 138, 142, 146 are free to move with respect to each other such that: the third floor piece 146 may rotate about the axis 158 within the slot 150 in the first floor piece 138; the third floor piece 146 may translate within the slot 150 of the first floor piece 138; and the second floor piece 142 may rotate relative to both the first floor piece 138 and the third floor piece 146. As such, the patient support 114 coupled to the third floor piece 146 moves radially between a center position (e.g., FIGS. 19A, 0 mm offset), an intermediate position (FIG. 19B), and a maximum offset from center position (e.g., FIG. 19C, approximately 350 mm offset).

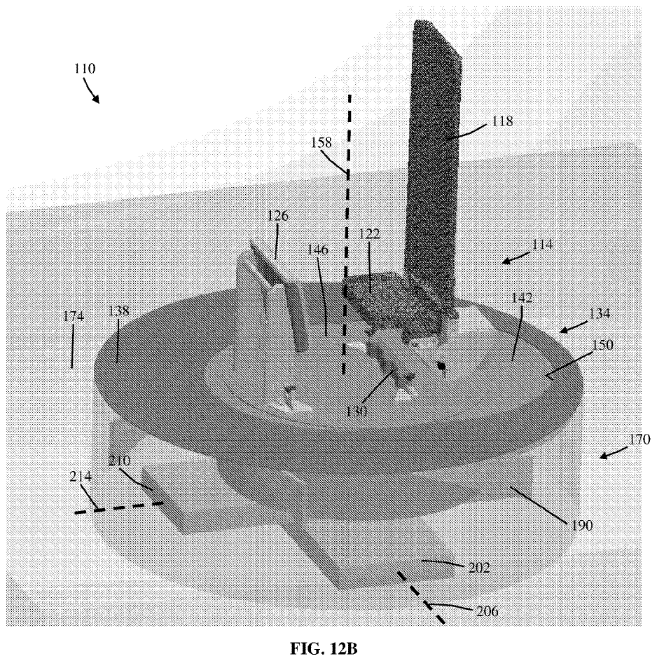

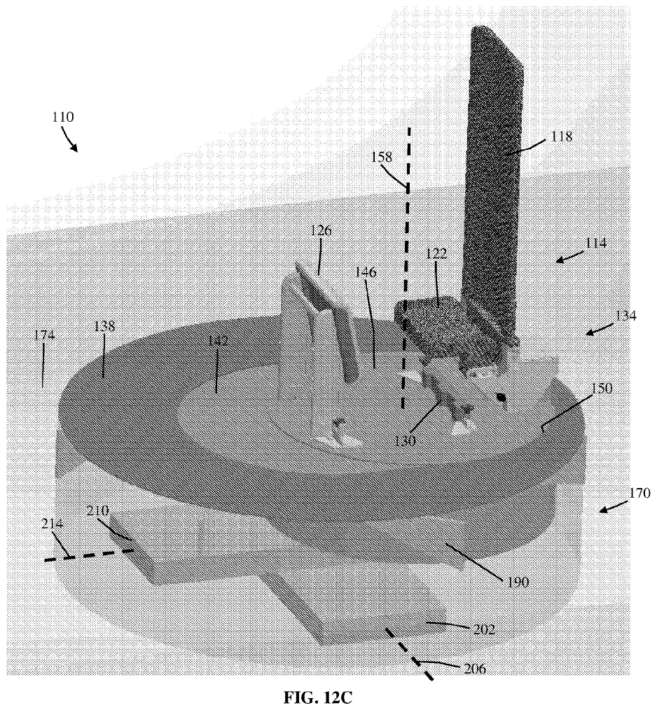

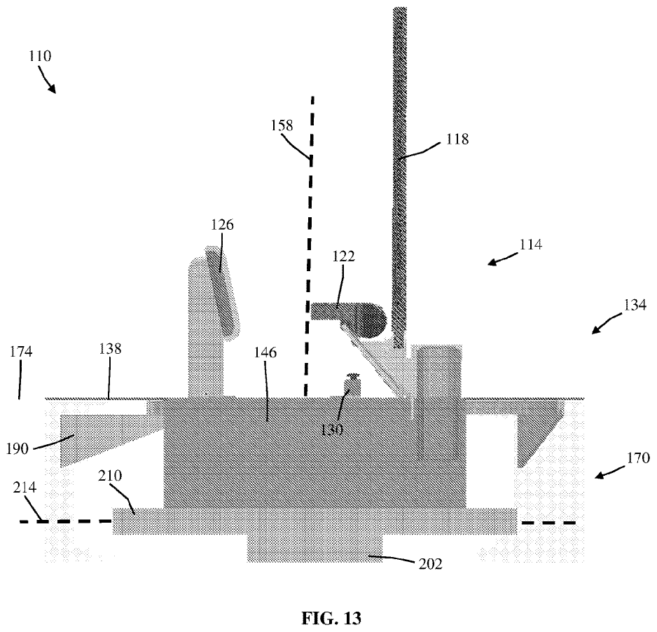

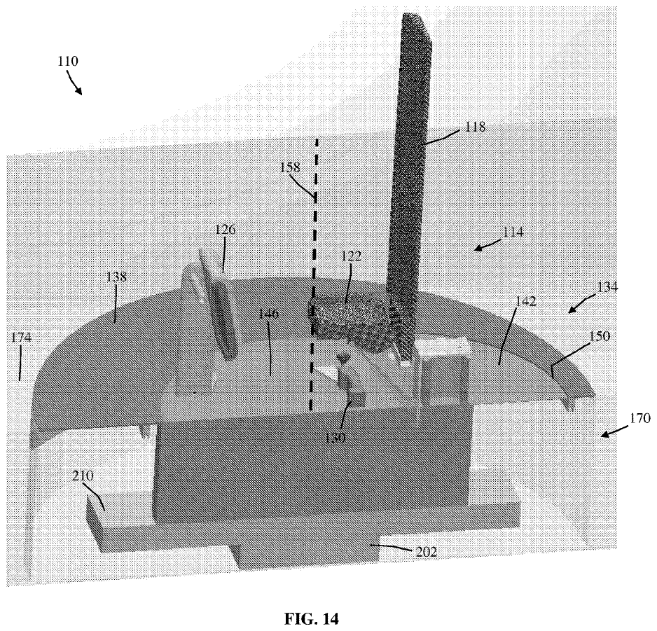

With reference to FIGS. 13 and 14, the floor assembly 134 covers an approximately 1100 mm diameter pit 170 in a room floor 174.

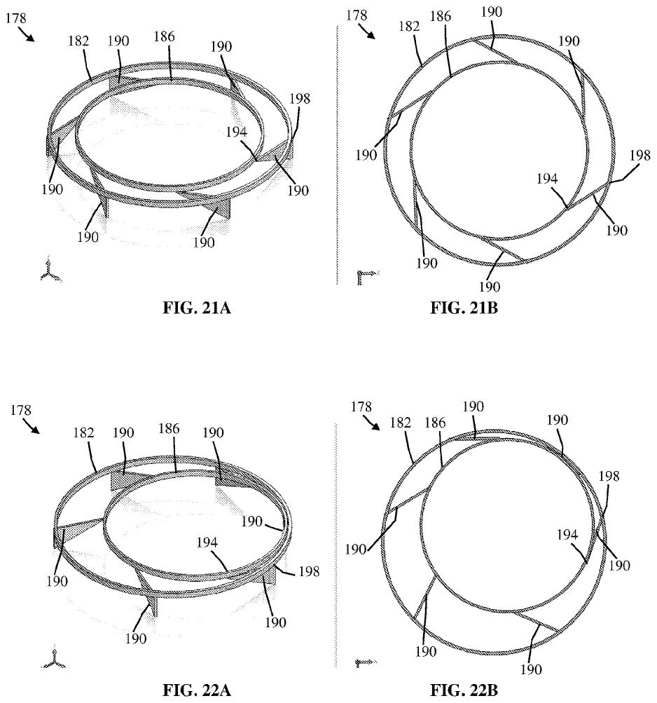

With reference to FIGS. 21A, 21B, 22A, and 22B, the patient positioning system 110 further includes a support assembly 178 coupled to the floor assembly 134. In the illustrated embodiment, the support assembly 178 includes an outer frame 182, and inner frame 186 coupled to the second floor piece 142, and a plurality of brackets 190 positioned between the inner frame 186 and the outer frame 182. In the illustrated embodiment, the inner frame 186 is an inner ring and the outer frame 182 is an outer ring.

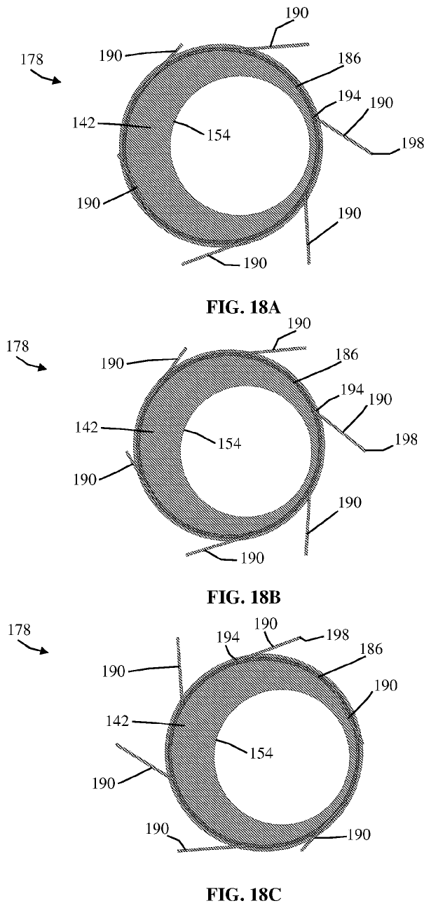

With reference to FIGS. 18A-18C, each of the plurality of brackets 190 is pivotably coupled to the inner frame 186 at a first end 194 of each bracket 190. With reference to FIGS. 19A-19C, each of the plurality of brackets 190 is pivotably coupled to the outer frame 182 at a second end 198 opposite the first end 194. In the illustrated embodiment, each of the plurality of brackets 190 is angled. In other words, the bracket 190 is narrower at the first end 194 than at the second end 198. In the illustrated embodiment, the brackets 190 transfer the loads from the inner region to the outer frame 182. In the illustrated embodiment, the brackets 190 are hinged at both the first end 194 and the second end 198. As such, the support assembly 178 provides vertical support to the second floor piece 142 at all possible positions of the patient support 114.

In some embodiments, the patient positioning system 110 includes an actuator coupled to the second floor piece 142 that is configured to move the second floor piece 142. In some embodiments, the floor assembly may have a possible stall position at the center for the second floor piece 142 depending upon translation of the overall system. Therefore, second floor piece 142 may utilize, in some embodiments, a preliminary knowledge of which direction the translation will be and an active mechanism (e.g., the actuator) to move the second floor piece 142 in the correct direction. In some embodiments, the actuator includes a motor and a gear assembly or other any other suitable active mechanism.

With reference to FIGS. 12A-12C, the patient positioning system 110 further includes a first linear translation stage 202 aligned along a first translation axis 206 and a second linear translation stage 210 aligned along a second translation axis 214. In the illustrated embodiment, the first linear translation stage 202 and the second linear translation stage 210 are positioned within the pit 170 formed in the room floor 174. In the illustrated embodiment, the first linear translation stage 202 is coupled to the third floor piece 146. The first linear translation stage 202 is configured to move the third floor piece 146 along the first translation axis 206. In the illustrated embodiment, the second linear translation stage 210 is coupled to the third floor piece 146. The second linear translation stage 210 is configured to move the third floor piece 146 along the second translation axis 214. In some embodiments, the second translation axis 214 is perpendicular to the first translation axis 206.

### Systems

The technology provides embodiments of systems. For example, the technology provides multi-axis medical imaging systems. In some embodiments, the medical imaging system is a computerized tomography (CT) system, a magnetic resonance imaging (MRI) system, a positron emission tomography (PET) system, a single photon emission computerized tomography (SPECT) system, a photon counting computed tomography system, or a portal imaging system or scanned projection radiography imaging system. While the technology is described for exemplary embodiments wherein the medical imaging system is a computerized tomography (CT) system, the technology is not limited to a CT scanning system and embodiments are to be understood to include other types of medical imaging systems.

In some embodiments, systems comprise a multi-axis medical imaging apparatus as described herein and software components and/or hardware components structured to rotate the gantry and/or to translate the scanner ring. In some embodiments, systems comprise software components structured to perform a method as described herein. In some embodiments, systems comprise a multi-axis medical imaging apparatus, software for obtaining (e.g., recording, acquiring) a medical image, and software for controlling gantry rotation and scanner ring translation.

In some embodiments, systems comprise a multi-axis medical imaging apparatus as described herein and a controller. In some embodiments, the medical imaging source and detector communicate with the controller. In some embodiments, the controller activates the medical imaging source and collects the image projections from the detector. In some embodiments, the controller controls movement of the medical imaging source and detector in opposition around the scanner ring. In some embodiments, the controller communicates with a camera (e.g., a horizontal camera and/or a vertical camera) positioned to obtain an elevational image and/or a plan image of a region occupied by a patient. In some embodiments, the controller communicates with a graphic display terminal for providing output images such as tomographic images, positioning information, and user input devices such as a keyboard for receiving instructions from a user. In some embodiments, the controller has a general computer architecture including one or more processors communicating with a memory for the storage of non transient control programs (e.g., to store tomographic projection sets and resulting tomographic images).

In some embodiments, systems comprise a multi-axis medical imaging apparatus comprising one or more cameras (e.g., a scanner ring comprising one or more cameras). In some embodiments, the cameras record images that are subsequently processed by software (e.g., configured to perform image recording, image analysis, image storage, image manipulation, and/or image comparison methods) and/or hardware components (e.g., microprocessors, graphics processors, communications buses configured to communicate, record, analyze, store, manipulate, and/or compare images) of the imaging subsystem.

In some embodiments, systems comprise a multi-axis CT scanner as described herein and software components and/or hardware components structured to rotate the gantry and/or to translate the scanner ring. In some embodiments, systems comprise a multi-axis CT scanner as described herein and software components and/or hardware components structured to rotate and/or translate the patient support. In some embodiments, systems comprise software components structured to perform a method as described herein.

In some embodiments, systems comprise a multi-axis CT scanner, software for obtaining (e.g., recording, acquiring) a CT scan, and software for controlling gantry rotation and scanner ring translation.

In some embodiments, systems comprise a multi-axis CT scanner as described herein, a patient in an upright (e.g., vertical (e.g., substantially and/or essentially vertical position)), and a user who interacts with controls structured to move the multi-axis CT scanner and acquire CT scans of said patient or a portion thereof.

In some embodiments, systems comprise a therapy beam apparatus, software for controlling the therapy beam, and software for controlling the patient positioning system.

### Uses

In some embodiments, the technology provided herein finds use in medical, clinical, and research settings. For example, in some embodiments, the technology finds use in imaging a biological system, e.g., an organism (e.g., an animal, a human), organ, tissue, and/or cell. In some embodiments, the technology finds use in imaging a head, neck, lungs, heart, circulatory system (e.g., arteries and/or veins), abdomen, pelvic region, gastrointestinal system, axial skeleton (e.g., spine), kidneys, and/or extremities. For example, in some embodiments, the technology finds use in diagnosing and/or treating a disease and/or injury. For example, the technology finds use in preventive medicine, disease screening, disease diagnosis, disease treatment, and/or disease monitoring. For example, in some embodiments, the technology finds use in diagnosing and/or treating a cancer. In some embodiments, the technology finds use in imaging the chest, e.g., for diagnosis of pneumothorax, emphysema, cardiomegaly, fibrosis, diaphragmatic hernias, empyema, atelectasis, pneumonia, pulmonary edema, pulmonary hemorrhage, primary lung malignancy, or a metastatic disease. In some embodiments, the technology finds use in diagnosing and/or treating a calcification, bone trauma, hemorrhage, edema, infarction, and/or tumor. The technology also finds use in research settings, e.g., to image an animal, human, organ, or tissue for research uses. The technology also finds use in veterinary medical settings, e.g., to image an animal, organ, or tissue for diagnosis and/or treatment. In some embodiments, the technology finds use in industrial uses, e.g., to image a non-biological object, e.g., to identify characteristics of construction, material defects, internal contents, etc., without breaking or otherwise disrupting the non-biological object.

Although the disclosure herein refers to certain illustrated embodiments, it is to be understood that these embodiments are presented by way of example and not by way of limitation. All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

What is claimed is:

1. A patient positioning system comprising:
an opening formed in a floor;
a carriage movable with respect to the opening; and
a stack of plates coupled to the carriage; wherein the stack of plates includes:
a first plate,
a second plate,
a spacer positioned between the first plate and the second plate, and
a key coupled between the first plate and the second plate;
wherein the first plate is slidable with respect to the second plate.

2. The patient positioning system of claim 1, wherein the key is positioned within a slot formed in the second plate, and the key is fastened to the first plate.

3. The patient positioning system of claim 2, wherein the spacer is positioned within a groove formed in the second plate.

4. The patient positioning system of claim 3, wherein the slot is on a first side of the second plate and the groove is on a second side of the second plate.

5. The patient positioning system of claim 1, wherein the carriage includes a drive plate, and the drive plate is coupled to the first plate.

6. The patient positioning system of claim 1, wherein the opening is entirely covered.

7. The patient positioning system of claim 1, wherein the stack of plates further includes a third plate, wherein the second plate is positioned between the first plate and the third plate.

8. The patient positioning system of claim 7, wherein the key is a first key, and the stack of plates further including a second key coupled between the second plate and the third plate.

9. The patient positioning system of claim 8, wherein the first key is offset from the second key.

10. The patient positioning system of claim 1, wherein the spacer at least partially overlaps the key.

11. The patient positioning system of claim 1, wherein the spacer is positioned within a groove formed in the second plate.

12. The patient positioning system of claim 1, further including a corner plate coupled to the stack of plates and configured to move between a retracted position and an extended position.

13. The patient positioning system of claim 12, further including a cover at least partially defining the opening, and wherein the corner plate is positioned between the stack of plates and the cover.

14. The patient positioning system of claim 1, wherein the stack of plates is a first stack of plates, and the patient positioning system further includes a second stack of plates.

15. The patient positioning system of claim 14, wherein the carriage is positioned between the first stack of plates and the second stack of plates.

16. The patient positioning system of claim 14, wherein the first stack of plates and the second stack of plates adjusts in response to movement of the carriage within the opening.

17. The patient positioning system of claim 16, further including a first corner plate coupled to the first stack of plates and a second corner plate coupled to the second stack of plates.

18. The patient positioning system of claim 1, further comprising a cover at least partially defining the opening, wherein the cover has a circular outer profile.

19. The patient positioning system of claim 1, further comprising a patient support coupled to the carriage.

20. The patient positioning system of claim 1, further comprising an actuator configured to move the carriage, wherein the actuator is positioned below the floor.

21. A patient positioning system comprising:
a floor assembly including:
a first floor piece with a slot,
a second floor piece with an aperture positioned offset from a center of the of the second floor piece, and
a third floor piece positioned within the aperture; and
a patient support coupled to the third floor piece;

22. The patient positioning system of claim 21, wherein the third floor piece defines an axis and the third floor piece is rotatable about the axis with respect to the first floor piece.

23. The patient positioning system of claim 21, wherein the third floor piece is positioned within the slot.

24. The patient positioning system of claim 23, wherein the third floor piece is movable within the slot relative to the first floor piece.

25. The patient positioning system of claim 21, wherein the second floor piece is rotatable with respect to the first floor piece and the third floor piece.

26. The patient positioning system of claim 21, wherein the third floor piece includes a lip and the second floor piece is at least partially positioning on the lip.

27. The patient positioning system of claim 21, further comprising a support assembly coupled to the floor assembly, wherein the support assembly includes an outer frame, an inner frame coupled to the second floor piece, and a plurality of brackets positioned between the inner frame and the outer frame.

28. The patient positioning system of claim 27, wherein the inner frame is an inner ring and the outer frame is an outer ring.

29. The patient positioning system of claim 27, wherein each of the plurality of brackets is angled.

30. The patient positioning system of claim 27, wherein each of the plurality of brackets is pivotably coupled to the inner frame at a first end and pivotably coupled to the outer frame at a second end opposite the first end.

31. The patient positioning system of claim 21, further including an actuator coupled to the second floor piece and configured to move the second floor piece.

32. The patient positioning system of claim 21, wherein the first floor piece is at least partially positioned on the second floor piece.

33. The patient positioning system of claim 21, further comprising a first linear translation stage coupled to the third floor piece and configured to move the third floor piece along a first translation axis and a second linear translation stage coupled to the third floor piece and configured to move the third floor piece along a second translation axis.
